(19) United States
(12) Patent Application Publication
(10) Pub. No.: US 2024/0196507 A1
(43) Pub. Date: Jun. 13, 2024

(54) METHODS AND APPARATUS FOR GENERATING ATMOSPHERIC PRESSURE, LOW TEMPERATURE PLASMA USABLE FOR AFFECTING FLUID FLOW

(71) Applicant: TellaPure, LLC, Purcellvile, VA (US)

(72) Inventors: Richard P. Lawless, Great Falls, VA (US); Edgar Bryan Hill, Ashburn, VA (US); Dawn Price, Great Falls, VA (US); Hidetaka Noda, Tokyo (JP); Akitoshi Okino, Tokyo (JP)

(73) Assignee: TellaPure, LLC, Purcellville, VA (US)

(21) Appl. No.: 17/868,730

(22) Filed: Jul. 19, 2022

Related U.S. Application Data
(60) Provisional application No. 63/355,633, filed on Jun. 26, 2022.

(51) Int. Cl. H05H 1/24; A61L 9/04; A61L 9/12; A61L 9/20; A61L 9/22

(52) U.S. Cl. CPC H05H 1/2406; A61L 9/046; A61L 9/122; A61L 9/20; A61L 9/22; A61L 2209/12; A61L 2209/15; H05H 2245/10; H05H 2245/36

(57) ABSTRACT

Embodiments relate to a plasma generator including a dielectric layer elongated in a longitudinal direction that extends 0.01 mm-2 mm in a thickness direction perpendicular to the longitudinal direction. The dielectric layer defines first and second planar surfaces that are separated in the thickness direction. A first electrode is disposed along a first portion of the first planar surface. A second electrode is disposed along a second portion of the second planar surface, such that at least a part of the first and second portions are separated in the longitudinal direction of the dielectric layer. A power supply is configured to supply electrical power to the first and second electrodes at a predetermined voltage and frequency, wherein plasma is generated adjacent each of the first and second electrodes and along the first and second surfaces of the dielectric layer other than the first and second portions.

### BACKGROUND

Some embodiments relate to methods and apparatus for generating, dispersing, ejecting, controlling, and/or using free radicals. Some of these embodiments more specifically relate to methods and apparatus for generating the free radicals via atmospheric pressure, low-temperature plasma (hereinafter “cold plasma” or “non-equilibrium plasma” or “stable plasma” or “plasma”). Some embodiments use these free radicals to affect or breakdown molecules in order to neutralize harmful matter in an air conditioning system or air duct system. Some other embodiments are directed to generating atmospheric pressure, low temperature plasma usable for affecting fluid flow, such as, for example, changing the vector of the flow of gases including but not limited to air.

Cold plasma is plasma where the temperatures of the individual constituents are different from each other. Electrons exist at higher temperatures (more than 10,000K) and neutral atoms can exist at room temperature. However, the density of the electrons in cold plasma is very low compared to the density of the neutral atoms. In a laboratory, cold plasmas are generally produced by applying electrical energy to different inert gases. This production can be performed at room temperature and at atmospheric pressure, obviating costly instruments and thereby reducing the overall costs associated with making cold plasma.

On a molecular level, cold plasma can be produced by moving accelerated electrons through certain gasses, e.g., helium or air. These electrons impact the atoms and molecules with so much energy that they separate the outermost electrons of the atoms and molecules in the gas, thereby creating a soupy mixture of free electrons and free ions. The gas remains at approximately room temperature because the energy required to separate the electrons from their atoms quickly dissipates, leaving the gas ions cool. The relatively low-density plasma enables controlled ionization of the available gas, which generates little to no detectable sound.

On a practical level, cold plasma is produced based on dielectric-barrier discharge (DBD) technology, including surface dielectric-barrier discharge (hereinafter “plasma actuator” or “SDBD”), which is the electrical discharge between two electrodes separated by an insulating dielectric barrier. DBD has been referred to as silent (inaudible) discharge, ozone production discharge, or partial discharge. In the related art, DBD requires high voltage alternating current ranging from lower radio frequency to microwave frequencies. Plasma is produced by two electrodes with a dielectric layer between the electrodes to limit the current flow in the plasma. The dielectric layer that limits the current controls the rate of ionization of the gas. DBD constitutes a dry method of plasma production that does not generate wastewater or require drying of the material after treatment.

### SUMMARY

Creating stable cold plasma can be difficult because it involves balancing numerous factors. For example, changes in voltage, composition of gases entering the system, air flow rate, relative humidity, electrode and insulting layer physio-chemical characteristics, introduction of catalysts, synergetic technologies, etc., can impact the production and concentration of the reactive and non-radicalized species, ions, electrons, and ultraviolet photons.

However, the potential advantages of generating stable cold plasma are enormous, because the free electrons and free ions created thereby can be selectively transmitted to impact and break down molecules. In other words, it may be beneficial to use cold plasma in a controlled manner so as to generate, disperse, eject, control, or otherwise use free radicals to selectively impact and break down certain molecules. It may also be beneficial to use free radicals to affect or break down molecules into certain constituent elements, and then isolate or otherwise use some of the constituent elements. Thus, cold plasma can be used in various applications, including but not limited to air sterilization, odor neutralization, ozone generation, gas reforming including but not limited to methane and methanol, carbon capture, broad area low-level activation processes, substance creation, etc.

As one example, breaking down molecules of a microorganism (single-cell organism) will effectively terminate the organism, thereby operating to sterilize the area in which the microorganism existed, i.e., virus inactivation. In other words, this process can effectively inactivate viruses suspended in the air or on surfaces.

As another specific example, it may be beneficial to provide an air purification device using cold plasma, wherein a first electrode is disposed on the top surface of a dielectric layer, and a second electrode is disposed on the bottom surface of the same dielectric layer and to the side, so that the second electrode is not directly underneath the first electrode, by which a cold plasma can be generated on the bottom surface of the dielectric layer directly below the first electrode and on the top surface of the dielectric layer directly above the second electrode. The electrodes and dielectric layer can be formed into a long strip to enable air to contact the cold plasma generated on the surface of the dielectric to obviate viruses, pathogens, mold toxins, etc., that were disposed in the air.

The free radicals generated by cold plasma as discussed above can also be used to breakdown molecules in water and on solid surfaces. This technology may similarly be used to clean and sterilize areas and gases within internal combustion engines (ICE), waste plants, industrial mines, etc., such as by breaking down carbon dioxide into separate carbon and oxygen atoms. In these cases, oxygen would be released, and carbon captured or sequestered.

It may be especially beneficial to generate stable cold plasma with high concentrations of Reactive Oxygen Species (“ROS”) and Reactive Nitrogen Species (“RNS”) that are highly effective at inactivating numerous pathogens in the air, on surfaces, and in water. Additionally, reactive species, ions, and electrons are relevant, important, or crucial to decomposing dangerous compounds into non-reactive compounds, chemically inert substances, and basic elements.

Cold plasma can be used on a grand scale as an active filtration system, wherein a fluid (such as air), passes through an arrangement of plasma actuators where the air is sterilized. An active filtration system, such as an HVAC application, may include a number of belt-like plasma actuators, such as for example one or multiple 300 mm×10 mm×0.15 mm belt-like plasma actuators, producing a corresponding one or multiple plasma generating regions. A singular plasma actuator may process a certain amount of air (such as 50-100 cfm), and so some embodiments may use multiple plasma actuators (such as combining 6 plasma actuators) to process as much fluid as is required, e.g., 300-600 cfm. However, as indicated above, any number of actuators can be used as well as actuators of any sizes, such as 600 mm×12 mm×0.2 mm.

It may be beneficial to integrate the plasma actuator with air quality surveillance monitoring technology, or other air quality control and surveillance technology, which allows for active testing of indoor air quality to further control the plasma actuator operation. Some embodiments of the plasma actuator may produce small amounts of ozone, so the system can be configured to automatically cease operations if the output approaches CARB (0.05 ppm) or OSHA (0.1 ppm) standards (pre-determined limits). Some of these embodiments may also be configured to measure gas concentrations, such as carbon dioxide, carbon monoxide, particulate matter <2.5 microns (PM 2.5), PM 1.0, Formaldehyde, Volatile Organic Compounds (VOCs), Nitrogen Oxides (NOₓ)₂, etc.

It may be especially beneficial for the plasma actuator to be configured to operate using relatively low power. For example, in some embodiments, each plasma generator operates on less than 60 watts of electrical power, which is the equivalent of a single light bulb.

It may be beneficial to reduce the size of a plasma generating apparatus (also referred to as a “plasma actuator apparatus”) while maintaining performance such as air cleaning. Therefore, it may also be beneficial for a plasma actuator to be made extremely thin so that the plasma actuator can fit any space required for its use. In this case, special materials and designs disclosed below will have to be used to allow the plasma actuator to effectively generate a stable cold plasma while also decreasing the size footprint of the plasma actuator as much as possible.

It may also be beneficial to allow the plasma actuator to be applied to any surface with very little effort, as a fast and simple method of sterilizing fluid flowing over the surface. Multiple plasma actuators may be applied to the same surface, and the arrangement of actuators will depend on the specifications of the surface and the fluid flowing over the surface.

It may be especially beneficial to generate atmospheric pressure, low temperature plasma usable for affecting fluid flow, such as, for example, changing the vector of the flow of gases including but not limited to air. In other words, atmospheric pressure, low temperature plasma can be generated and ejected in a manner so as to change the direction of air flow. In some of these embodiments, air that is flowing in a direction generally toward a planar surface of the dielectric/electrodes of the plasma generator (i.e., perpendicular to the apparatus) can be redirected into a direction generally along a direction of elongation of the dielectric/electrodes, which constitutes generally a 90 degree change of direction. However, embodiments are intended to include or otherwise cover any amount or direction of change of air. In fact, embodiments are intended to include or otherwise cover changing the flow of any type of fluid including any type of gas, liquid, slurry, etc.

Thus, some embodiments relate to a plasma generator for generating atmospheric pressure, low temperature plasma, including: a dielectric layer, having a dielectric constant, that is elongated in a longitudinal direction, and that extends 0.01 mm-2 mm in a thickness direction that is perpendicular to the longitudinal direction, the dielectric layer defining a first planar surface and an opposing second planar surface that are separated in the thickness direction; a first electrode that is disposed along a first portion of the first planar surface; a second electrode that is disposed along a second portion of the second planar surface, such that at least a part of the first and second portions are separated in the longitudinal direction of the dielectric layer; and a power supply configured to supply electrical power to the first and second electrodes at a predetermined voltage and frequency, such that, based on the dielectric constant of the dielectric layer, atmospheric pressure, low temperature plasma is generated adjacent each of the first and second electrodes and along the first and second surfaces of the dielectric layer other than the first and second portions.

In some of these embodiments, the power supply is configured to generate the atmospheric pressure, low temperature plasma so as to change a flow of fluid into a direction generally in the longitudinal direction of the dielectric layer.

In some of these embodiments, the dielectric layer extends 0.02 mm-0.08 mm in the thickness direction.

In some of these embodiments, the dielectric layer extends approximately 0.05 mm in the thickness direction.

Some other embodiments are directed to a plasma generator for generating atmospheric pressure, low temperature plasma, including: a dielectric layer, having a dielectric constant, that is elongated in a longitudinal direction, and that extends 0.01 mm-2 mm in a thickness direction that is perpendicular to the longitudinal direction, the dielectric layer defining a first planar surface and an opposing second planar surface that are separated in the thickness direction; a first electrode that is disposed along a first portion of the first planar surface; a second electrode that is disposed along a second portion of the second planar surface, such that at least a part of the first and second portions are separated in the longitudinal direction of the dielectric layer; an insulator that covers the second electrode and the second planar surface other than the second portion; and a power supply configured to supply electrical power to the first and second electrodes at a predetermined voltage and frequency, such that, based on the dielectric constant of the dielectric layer, atmospheric pressure, low temperature plasma is generated adjacent the first electrode proximate along the first surface of the dielectric layer other than the first portion.

In some of these embodiments, the power supply is configured to generate the atmospheric pressure, low temperature plasma so as to change the fluid flow into a direction generally in the longitudinal direction of the dielectric layer.

In some of these embodiments, the dielectric layer extends 0.02 mm-0.08 mm in the thickness direction.

In some of these embodiments, the dielectric layer extends approximately 0.05 mm in the thickness direction.

In some of these embodiments, the first electrode defines a groove in the longitudinal direction of the dielectric layer, so as to define first and second separated sections, and such that atmospheric pressure, low temperature plasma is generated proximate the groove.

Some other embodiments are directed to an apparatus for receiving a fluid, communicating atmospheric pressure, low temperature plasma to the fluid to result in treated fluid, and supplying the treated fluid to a space. The apparatus includes: a plasma generator for generating atmospheric pressure, low temperature plasma, the plasma generator including: a dielectric layer, having a dielectric constant, that is elongated in a longitudinal direction, and that extends 0.01 mm-2 mm in a thickness direction that is perpendicular to the longitudinal direction, the dielectric layer defining a first planar surface and an opposing second planar surface that are separated in the thickness direction; a first electrode that is disposed along a first portion of the first planar surface; a second electrode that is disposed along a second portion of the second planar surface, such that at least a part of the first and second portions are separated in the longitudinal direction of the dielectric layer; an insulator that covers the second electrode and the second planar surface other than the second portion; and a power supply configured to supply electrical power to the first and second electrodes at a predetermined voltage and frequency, such that, based on the dielectric constant of the dielectric layer, atmospheric pressure, low temperature plasma is generated adjacent the first electrode proximate along the first surface of the dielectric layer other than the first portion. The apparatus also includes: a housing that houses the plasma generator and that defines an entry aperture for enabling entry of the received fluid, and an exit aperture for enabling exit of the treated fluid into the space; and a fan configured to direct the received fluid to the plasma generator and through the exit aperture into the space.

Some of these embodiments further include: a heat exchanger that changes a temperature of the received fluid.

Some of these embodiments further include: a UV emitter and an ultrasonic oscillator that are disposed proximate the plasma generator and configured to facilitate generation of the atmospheric pressure, low temperature plasma.

Some of these embodiments further include: an ozone filter disposed proximate the exit aperture to impede passage of ozone in the treated fluid

### BRIEF DESCRIPTION OF FIGURES

Each figure describes basic features of various methods and apparatuses for generating and/or dispersing free radicals and for separating ionized molecules. Various exemplary aspects of the systems and methods will be described in detail, with reference to the following figures, wherein:

FIG. 1 is a cross-sectional schematic depicting basic features and effects of a plasma actuator discharge portion 100A according to an exemplary embodiment.

FIG. 2 is a cross-sectional schematic depicting basic features and effects of a plasma actuator discharge portion 100A according to an exemplary embodiment.

FIG. 3 is a top schematic view showing an exemplary configuration of a plasma actuator apparatus 100.

FIG. 4 is a schematic cross-sectional view of an exemplary embodiment of the discharge portion 100A of the plasma actuator apparatus 100 of FIG. 3.

FIG. 5 is a partial enlarged view of the discharge portion 100A of FIG. 4.

FIG. 6 is a partial top plan view of the discharge portion 100A according to an exemplary embodiment of the plasma actuator apparatus 100.

FIG. 7 is a schematic cross-sectional view of the discharge portion 100A of FIG. 6.

FIG. 8 is a partial enlarged view of the discharge portion 100A of FIG. 7.

FIG. 9 is a cross-sectional schematic view of an air conditioner comprising a plasma actuator device 100 according to an exemplary embodiment.

FIG. 10 is a perspective view schematically showing a portion of the air conditioner of FIG. 9.

FIG. 11 is a front schematic view of an exemplary embodiment of a plasma actuator structure 400A.

FIG. 12 is a perspective schematic view of the plasma actuator structure 400A of FIG. 11.

FIG. 13 is a side schematic view of the plasma actuator structure 400A of FIG. 11.

FIG. 14 is a partial enlarged side schematic view of the plasma actuator structure 400A of FIG. 11.

FIG. 15 is a partial enlarged side schematic view of the plasma actuator structure 400A of FIG. 11.

FIG. 16 is a top schematic view of an exemplary embodiment of a plasma actuator structure 400B.

FIG. 17 is a perspective schematic view of the plasma actuator structure 400B of FIG. 16.

FIG. 18 is a front schematic view of the plasma actuator structure 400B of FIG. 16.

FIG. 19 is a front schematic view of an exemplary embodiment of a plasma actuator structure 400B.

FIG. 20 is a front schematic view of an exemplary embodiment of a plasma actuator structure 400B.

FIG. 21 is a front schematic view of an exemplary embodiment of a plasma actuator structure 400B.

FIG. 22 is a top schematic view of an exemplary embodiment of a plasma actuator structure 400B.

FIG. 23 is a front schematic view of an exemplary embodiment of a plasma actuator structure 4000.

FIG. 24 is a front schematic view of an exemplary embodiment of a plasma actuator structure 4000.

FIG. 25 is a side schematic view of the plasma actuator structure 4000 of FIG. 24.

FIG. 26 is a perspective schematic view of an exemplary embodiment of a plasma actuator structure 400D.

FIG. 27 is a front schematic view of the plasma actuator structure 400D of FIG. 26.

FIG. 28 is a perspective schematic view of an exemplary embodiment of a plasma actuator structure 400E.

FIG. 29 is a schematic showing the connection of an exemplary embodiment of a power supply unit 100B to an exemplary embodiment of a plasma discharge portion 100A.

### DETAILED DESCRIPTION

The Detailed Description is organized based on the following headings.
I. Definitions
II. Plasma Actuator
A. Overview of the Plasma Actuator
B. Applications
C. Detailed Explanation
D. Variations of Embodiments

### I. Definitions

It will be understood that, when an element is referred to as being “connected”, or “coupled” to another element, it can be directly connected or coupled to the other element or intervening elements that may be present. In contrast, when an element is referred to as being “directly connected” or “directly coupled” to another element, there are no intervening elements present. As used herein “and/or” includes any and all combinations of one or more of the associated listing items. Further, it will be understood that when an element is “presented” to an entity, it can be presented electronically to the entity through multiple intermediaries or elements of the system. In addition, it will also be understood that when an element is referred to as being “directly presented” to an entity, it is presented electronically through only one intermediary or element of the system. In addition, it will be understood that when an element is presented or directly presented to an entity the presentation may take place on an electronic screen separate from any or all previous electronic screens.

It will also be understood that, although the terms “first”, “second”, etc. may be used herein to describe various elements or components, these elements or components should not be limited by these terms. These terms are only used to distinguish one element or component from another element or component. Thus, a first element or component discussed below may be termed a second element or component without departing from the teachings of exemplary embodiments. Further, it will be understood that the use of “then”, when used to describe connecting two steps of a logical process, indicates that the steps may occur sequentially, but does not preclude the addition of intermediary steps or elimination of one of the steps without departing from the teachings of exemplary embodiments.

Exemplary embodiments are described herein with reference to logical process illustrations that are schematic illustrations of idealized embodiments (and intermediate structures) of exemplary embodiments. As such, variations from the sequence of the illustrations as a result, for exemplary, inclusion of intermediary steps, are to be expected. Thus, exemplary embodiments should not be construed as limited to the particular sequence of logical steps illustrated herein but are to include deviations in the sequence of the steps, for exemplary, from communication of electronic information to remote databases. Thus, the logical steps illustrated in the figures are schematic in nature and their sequence is not intended to limit to scope of exemplary embodiments.

The terminology used herein is for the purpose of describing particular embodiments only and is not intended to be limiting of exemplary embodiments. As used herein, the singular forms “a”, “an”, and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which exemplary embodiments belong. It will be further understood that all terms, such as those defined in commonly used dictionaries, should be interpreted as having a meaning that is consistent with their meaning in the context of the relevant art and will not be interpreted in an idealized or overly formal sense unless expressly so defined herein. As used herein, expressions such as “at least one of”, when preceding a list of elements, modify the entire list of elements and do not modify the individual elements of the list.

Various electrical or electronic elements of the disclosed embodiments, including but not limited to the power supply and control circuitry, are intended to include or otherwise cover all processors, software, or computer programs capable of performing the various disclosed determinations, calculations, etc., for the disclosed purposes. For example, exemplary embodiments are intended to cover all software or computer programs capable of enabling processors to implement the disclosed processes. In other words, exemplary embodiments are intended to cover all systems and processes that configure a document operating system to implement the disclosed processes. Exemplary embodiments are also intended to cover any and all currently known, related art or later developed non-transitory recording or storage mediums (such as a CD-ROM, DVD-ROM, hard drive, RAM, ROM, floppy disc, magnetic tape cassette, etc.) that record or store such software or computer programs. Exemplary embodiments are further intended to cover such software, computer programs, systems and/or processes provided through any other currently known, related art, or later developed medium (such as transitory mediums, carrier waves, etc.), usable for implementing the exemplary operations disclosed above.

In accordance with the exemplary embodiments, disclosed computer programs can be executed in many exemplary ways, such as an application that is resident in the memory of a device or as a hosted application that is being executed on a server and communicating with the device application or browser via a number of standard protocols, such as TCP/IP, HTTP, XML, SOAP, REST, JSON and other sufficient protocols. The disclosed computer programs can be written in exemplary programming languages that execute from memory on the device or from a hosted server, such as BASIC, COBOL, C, C++, Java, Pascal, or scripting languages such as JavaScript, Python, Ruby, PHP, Perl or other sufficient programming languages.

Some of the disclosed embodiments include or otherwise involve data transfer over a network, such as communicating various inputs over the network. The network may include, for example, one or more of the Internet, Wide Area Networks (WANs), Local Area Networks (LANs), analog or digital wired and wireless telephone networks (e.g., a PSTN, Integrated Services Digital Network (ISDN), a cellular network, and Digital Subscriber Line (xDSL)), radio, television, cable, satellite, and/or any other delivery or tunneling mechanism for carrying data. The network may include multiple networks or subnetworks, each of which may include, for example, a wired or wireless data pathway. The network may include a circuit-switched voice network, a packet-switched data network, or any other network able to carry electronic communications. For example, the network may include networks based on the Internet protocol (IP) or asynchronous transfer mode (ATM), and may support voice using, for example, VoIP, Voice-over-ATM, or other comparable protocols used for voice data communications. In one implementation, the network includes a cellular telephone network configured to enable the exchange of text or SMS messages. Some of these and other embodiments utilize a Bluetooth network.

Examples of a network include, but are not limited to, a personal area network (PAN), a storage area network (SAN), a home area network (HAN), a campus area network (CAN), a local area network (LAN), a wide area network (WAN), a metropolitan area network (MAN), a virtual private network (VPN), an enterprise private network (EPN), Internet, a global area network (GAN), and so forth.

Central database systems may include a network server for communicating with the various remote computer systems. Communication to the network may be over the Internet, other networks, telephone, or other suitable means. The central database systems further include a central database and database server for storing and retrieving information. The network server can be operated by software that allows communication with the remote computer systems and transfers information to and from the database server for maintenance of the database and for providing patient-specific information.

Reference will now be made in detail to embodiments, examples of which are illustrated in the accompanying schematics, wherein like reference numerals refer to the like elements throughout. In this regard, the present embodiments may have different forms and should not be construed as being limited to the descriptions set forth herein.

Accordingly, the embodiments are merely described below, by referring to the figures, to explain exemplary embodiments of the present description. It is also important to note that various elements and features may be used interchangeably among the different disclosed embodiments, and so the various elements and features may be combined to achieve new embodiments.

### II. Plasma Actuator

### A. Overview of the Plasma Actuator

Embodiments are intended to include or otherwise cover a variety of structures and processes for generating cold plasma for any possible advantageous use, e.g., so that the free electrons and free ions created thereby can be selectively transmitted to impact and break down molecules. Some of the disclosed plasma actuator embodiments constitute long strip-like surface dielectric barrier plasma discharge units or “plasma actuators”; however, other embodiments can cover other types of units.

Some of the generated plasmas as disclosed herein are atmospheric pressure, low-temperature plasma (hereinafter “cold plasma” or “non-equilibrium plasma” or “stable plasma” or “plasma”). However, embodiments are intended to include or otherwise cover other types of plasmas that may be beneficial.

Some of the disclosed plasma actuators include a first electrode that is disposed on at least part of the top surface of a dielectric layer and a second electrode that is disposed on the bottom surface of the same dielectric layer and to the side, so that the second electrode is not directly underneath the first electrode. In some embodiments, the electrodes and the dielectric layer are long strips. However, embodiments are intended to cover or otherwise include other structures for generating the cold plasma, such as including but not limited to one of the electrodes being covered (e.g., in part) with a dielectric layer, or alternatively multiple dielectric layers being disposed between a pair of electrodes. Moreover, some embodiments can include multiple plasma actuators “stacked” on top of one another.

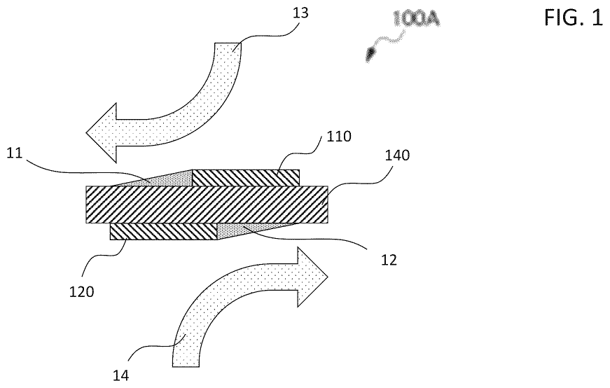

FIG. 1 is a schematic depicting basic features and effects of the discharge portion 100A of a plasma actuator according to an exemplary embodiment. A first electrode 110 is disposed on the top surface of a dielectric layer 140, and a second electrode 120 is disposed on the bottom surface of the dielectric layer 140. The electrodes 110, 120 and the dielectric layer 140 may be formed into strips where the length of the strips may be greater than the width of the strips defined by the side-on view shown in FIG. 1. When a specified electrical signal is supplied to one or both electrodes 110,120, a plasma 11 is formed on the top surface of the dielectric layer 140 directly above the second electrode 120, and another plasma 12 is formed on the bottom surface of the dielectric layer 140 directly below the first electrode 110.

The electrodes 110,120, when supplied with a specified electrical signal, can create an electrical field that accelerates the plasma 11,12 in a specified direction. In some embodiments, the plasma 11 can be accelerated parallel to the surface of the dielectric layer 140 and away from the top electrode 110, generating an induced flow 13 within a surrounding fluid. Similarly, the plasma 12 can be accelerated parallel to the surface of the dielectric layer 140 and away from the bottom electrode 120, generating an induced flow 14 within a surrounding fluid.

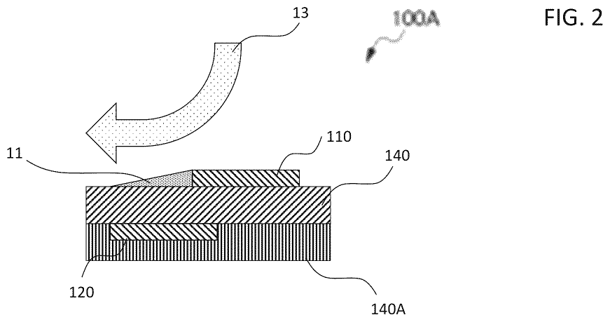

In FIG. 2, a similar structure to FIG. 1 is presented, wherein a first electrode 110 is disposed on at least part of the top surface of a dielectric layer 140, and a second electrode 120 is disposed on at least part of the bottom surface of the dielectric layer 140. The electrodes 110, 120 and the dielectric layer 140 may be formed into strips where the length of the strips may be greater than the width of the strips defined by the side-on view shown in FIG. 2. However, when the second electrode 120 is covered by a second dielectric layer 140A, only one plasma 11 is formed on the top surface of the dielectric layer 140 directly above the second electrode 120. The dielectric layer 140A may also be formed into a strip where the length of the strip extends greater than the width of the strips defined in FIG. 2. Just as in FIG. 1, the electrodes 110,120, when supplied with a specified electrical signal, can create an electrical field that accelerates the plasma 11 in a specified direction. In some embodiments, the plasma 11 can be accelerated parallel to the surface of the dielectric layer 140 and away from the top electrode 110, generating an induced flow 13 within a surrounding fluid.

In some of these embodiments, stable cold plasma 11,12 is generated based on the following factors: 1) geometric placement of electrodes; 2) voltage of AC electrical power supplied to one or both electrodes; and 3) frequency of electrical power supplied to one or both of the electrodes. However, other factors can affect the stable cold plasma generation of some embodiments, including the materials from which the electrodes and dielectric layers are formed, the relative permittivity and strength of the dielectric layers, etc. For example, in order to generate stable cold plasma, the output voltage can be controlled based on certain structural parameters of the plasma generator, such as the horizontal distance separating the electrodes or the horizontal distance where the electrodes overlap, the materials from which the electrodes are formed, the strip length and thickness of the electrodes and dielectric layers, etc.

In addition, embodiments are intended to cover or otherwise include any combination of the above factors that achieve the generation and acceleration of stable cold plasma, including but not limited to the exemplary ranges specifically disclosed herein. Still further, embodiments are intended to include or otherwise cover any methods, processes, or structures for supplying, varying or otherwise modifying the electrical power to the electrodes having appropriate voltages and frequencies for generating the stable cold plasma.

A few exemplary plasma generator embodiments are disclosed below, however as indicated above, embodiments are intended to cover many different variances in structure and function from the specific elements and processes disclosed below.

### B. Applications

Embodiments are intended to cover various structures of plasma generators for creating or otherwise generating free electrons and free ions, such as to selectively transmit the generated free electrons and free ions to impact and break down molecules for any useful purpose. Embodiments are also intended to be configured for use with any advantageous or otherwise beneficial applications of the stable cold plasma and the free electrons and free ions generated thereby. For example, embodiments are intended to include or otherwise cover any advantageous or otherwise beneficial application of using the generated free electrons and free ions to break down or otherwise modify molecules.

Embodiments are also intended to be configured for use with any advantageous or otherwise beneficial applications of the acceleration of the generated ions and free electrons and the accompanying induced flow in a surrounding fluid. For example, embodiments are intended to include or otherwise cover any advantageous or otherwise beneficial application of using the induced flow to generate vortices for the purpose of achieving higher mixing of generated reactive species and surrounding fluid.

For example, embodiments can be used in various contexts related to disinfection, including viral inactivation. This usage may be especially poignant based on the fact that droplet transmission is associated with the spread of coronavirus and similar pathogens including other viruses, pathogenic bacteria, mold, poisons, gases, mycotoxins (aerosol infection) constitute one of the main routes of infection. Thus, it may be beneficial to structure or otherwise use the plasma actuator embodiments disclosed herein to contact air that may include such pathogens and thereby sterilize the air to reduce, minimize, or prevent the transmission and spread of the pathogens.

Embodiments are intended to include or otherwise cover any structures or processes that enable or otherwise adapt the disclosed plasma actuators for such sterilization usages. For example, the disclosed plasma actuators can be adapted to operate in conjunction with equipment configured to supply air to the generated plasma, and then export the sterilized air back into the environment in which the air was originally obtained.

For some of these embodiments that operate as air sterilizers, additional structures may be provided for enhanced sterilization, such as apparatus for increasing turbulence or vorticity of air before flowing over the surface of the plasma actuator. The increased turbulence or vorticity increases the contact rate of the air with the electrons and ions to enhance sterilization.

In some embodiments, the arrangement or structure of the plasma actuators is enough to generate one or multiple vortices to enhance the mixing of the air with the generated reactive species, allowing a greater contact rate between the generated reactive species and harmful structures within the air. The higher contact rate enables a higher inactivation rate of the harmful structures, enhancing sterilization of the air.

However, embodiments are intended to cover or otherwise include any other useful application of creating or otherwise generating free electrons and free ions, including but not limited to selectively transmit the generated free electrons and free ions to impact and break down molecules.

### C. Detailed Explanation

The plasma actuator discharge portion 100A of FIG. 1 is disclosed above at a high level and will be discussed in much more detail below.

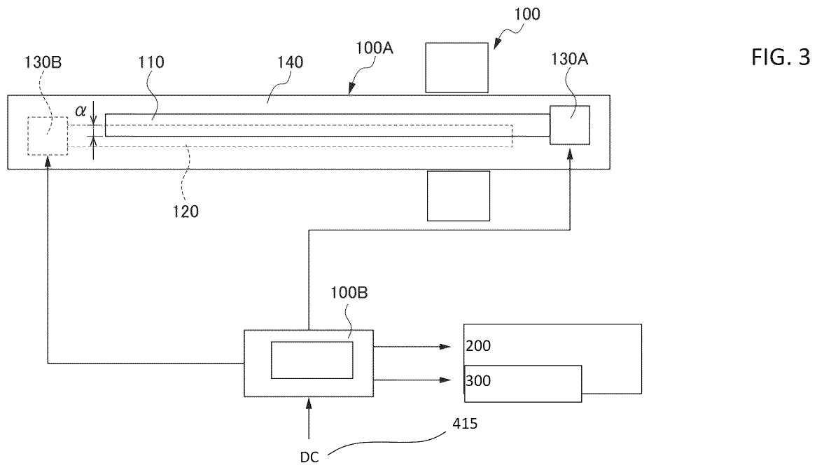

A top-view of an exemplary embodiment of the plasma actuator apparatus 100 is shown in FIG. 3, where the plasma actuator 100 comprises a discharge portion 100A and a power supply unit 100B. The discharge portion 100A is provided with a first electrode 110 on one surface and a second electrode 120 on the other surface along the longitudinal direction of the dielectric layer 140, which takes the shape of an elongated tape-like substrate.

The dielectric layer 140 is formed of a dielectric material such as various plastic resins in an elongated tape shape. The material, thickness, width, and length of the dielectric layer 140 are selected to be suitable for generating a surface dielectric barrier discharge between the electrodes.

The first electrode 110 and the second electrode 120 are each formed of an elongated, belt-like conductive material, for example, copper foil tape. The thickness of the electrodes 110 and 120 yields beneficial results between 0.01 mm and 2 mm, with even better results at 0.05 mm. The electrodes 110, and 120 may also be formed by applying a conductive coating material, for example, a coating of aluminum or copper by vapor deposition, to the surfaces of the dielectric layer 140. A first electrode 110 is formed on one surface of the dielectric layer 140, and a second electrode 120 is formed on the other surface of the dielectric layer 140.

Electrode terminals 130A, 130B are formed at one end of the first electrode 110 and the opposite end of the second electrode 120. The first electrode 110 and the second electrode 120 are connected to the power supply section 100B via electrode terminals 130A and 130B, respectively. The length, width, and thickness of the first electrode 110 and the second electrode 120 can be determined based on a multitude of interacting variables, including but not limited to the size of the dielectric layer 140, the material of the dielectric layer 140, the specification of the high-frequency voltage applied between the electrodes, and the dimensions of the electrodes 110, 120.

The power supply unit 1001B is a power supply unit that supplies a high-frequency voltage signal for generating dielectric barrier discharge between the first electrode 110 and the second electrode 120. The power supply unit 100B receives, for example, a DC 12 V power supply, from another power source 415 and converts it into a voltage signal having a frequency of a microwave band or an RF band by boosting and switching. Good results are yielded between 60 Hz and 300 GHz with better results between 10 kHz and 1 GHz and even better results at 220 kHz. The voltage signal may be, for example, an alternating sine wave, but is not limited thereto. The applied voltage yields beneficial results between 500 V and 15 kV, with better results between 2 kV and 9 kV, and better results still at 4 kV. The power supply unit 1001B may also provide power to a UV emitter 200 and an ultrasonic oscillator 300.

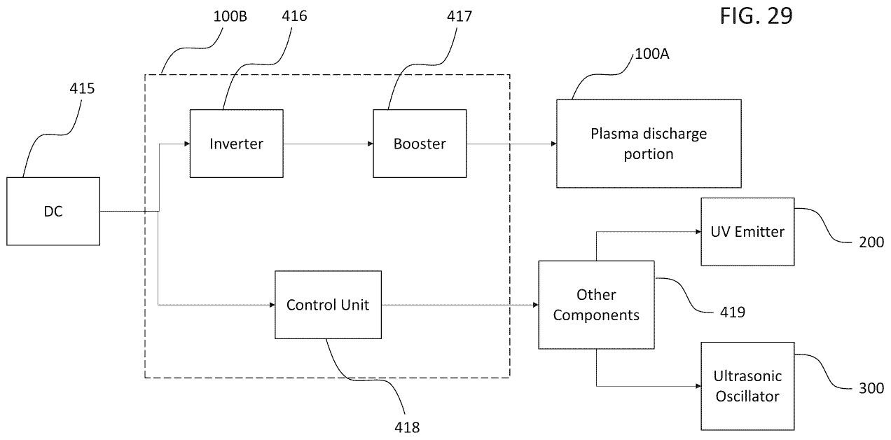

FIG. 29 shows an exemplary control configuration of the power supply unit 1001B. A power source 415 provides a direct current voltage to the power supply unit 1001B. The power supply unit 100B may include an inverter 416, which turns the DC signal into an AC signal with a specified frequency and shape, a booster 417, which increases the voltage of the AC signal from the inverter 416. The signal from the booster can connect to the plasma discharge portion 100A in the manner described in FIG. 3. The power supply unit 100B may also include a control unit 418, which provides the required power for other components 419, which may include a UV emitter 200 and an ultrasonic oscillator 300.

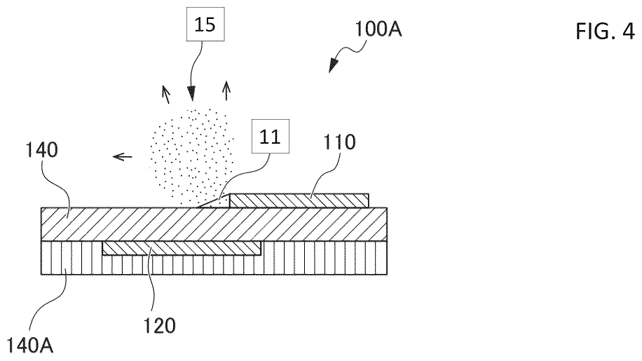

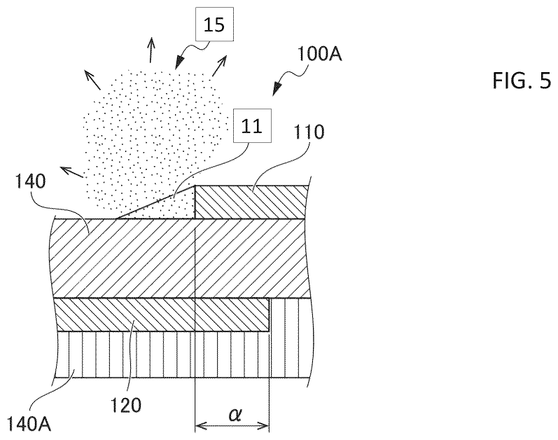

Next, the generation of atmospheric pressure low-temperature plasma in the discharge portion 100A will be described. FIG. 4 is a schematic cross-sectional view of the discharge portion 100A, and FIG. 5 is a partial enlarged view thereof. As shown in FIG. 5, the first electrode 110 and the second electrode 120 provided on both surfaces of the dielectric layer 140 are arranged so that they overlap each other by a width a when viewed in the width direction of the substrate 140. The second electrode 120 is molded by a cover layer 140A formed of a suitable insulating resin material or other dielectric material or insulating material, for example an electrically insulating epoxy resin or the same dielectric material as the substance used for the dielectric layer 140.

With such a configuration, in the discharge portion 100 A, when a high-frequency voltage is applied between the first electrode and the second electrode 120, a surface dielectric barrier discharge is generated between the first electrode 110 and the second electrode through the substrate 140, and an atmospheric pressure low-temperature plasma 11 is generated in the region next to the first electrode 110 and above the second electrode 120 on the surface of the dielectric layer 140. The atmospheric pressure low temperature plasma acts on the surrounding air and water vapor to produce Reactive Species (RS) 15 including various radicals such as singlet oxygen (¹O₂), ozone (O₃), hydroxyl radical (OH), superoxide anion radical (O₂⁻), hydroperoxyl radical (HO₂), and hydrogen peroxide (H₂O₂) as is known. The structure of microorganisms such as viruses and bacteria that come into contact with the reactive species 15 is destroyed in a very short time on the order of microseconds by contacting with the plasma discharge, and the viruses are inactivated and the microorganisms are sterilized by mixing with the multi-plasma gas containing the active oxygen species.

Further, as described above, since the discharge portion 100A as a whole has a tape-like structure in which very thin layers of the first electrode 110, the dielectric layer 140, the second electrode 120, and the cover layer 140A are adhered, the discharge portion 100A can be mounted in almost any position and facing in almost any desired direction, including towards or away from the flow of incoming fluid or ambient air, depending on the desired effect of inducing a flow direction or even forming vortices.

In some embodiments, a UV emitter 200 and an ultrasonic oscillator 300 can be added to assist in both the sterilization of harmful molecules in the air and the generation of the plasma itself in the plasma regions 11,12,16, and 17. The ultraviolet lamp 200 irradiates the air with light having a wavelength in the ultraviolet region mainly for sterilizing action. In some embodiments, the ultraviolet light can also be absorbed by the molecules in the plasma regions 11,12,16, and 17 to assist in generating the plasma by helping to ionize the molecules. A light emitting device used as a general sterilizing lamp can be used as the UV emitter 200. As the light source, for example, an ultraviolet LED lamp can be used. Bacteria and the like contained in the air flowing through the air passage 19 can be removed or inactivated by the ultraviolet light lamp 200. The ultrasonic oscillator 300 is a device that emits ultrasonic waves with a frequency on the order of several 10s of kHz at an appropriate output and serves to support the cleaning of air by damaging structures such as bacteria in the air or by promoting the mixing of various active species generated in the atmospheric pressure low-temperature plasma by the plasma actuator apparatus 100 with the air flowing therethrough.

The material used to bond the components to each other, including for example, the electrodes 120,130 to the dielectric layer 140, or even the cover layer 140A to one of the electrodes 130 and the dielectric layer 140, can be any known related art or later developed apparatus or method of bonding, including but not limited to adhesive, tape, epoxy, and glue. Embodiments are intended to include any mechanism of bonding, including by mechanical means, like rivets, bolts, or any other known related or later developed mechanical apparatus. It might also be beneficial for the bonding material to be temperature rated and to be able to keep a high bonding strength at temperatures above room temperature for long periods of time.

The width of the dielectric layer 140 yields good results between 5 mm and 60 mm with better results between 20 mm and 40 mm with even better results at 30 mm. The width of the electrodes 120,130 yields good results between 1 mm and 30 mm with better results between 5 mm and 20 mm with even better results at 15 mm. The overlap width a yields good results between −4 mm and 4 mm (where a negative overlap denotes a positive horizontal space between the electrodes 120,130), with better results between 1 and 3 mm and even better results at 2 mm. The length of the dielectric layer 140 and the electrodes 120,130 can be any length for the plasma actuator 100 to work as desired. The lengths can be tailored to fit any space required for their use. The material of the dielectric layer 140 can be chosen on the basis of its dielectric constant, which yields good results between 1 and 50, with even better results between 2 and 10 and still better results at 3. An example of the material of the dielectric layer 140 is polyimide film.

It may be beneficial for the dielectric layer 140 to be temperature rated and able to resist temperatures 380-400 degrees Celsius or greater. An example could be a polyimide film with a high-temperature rating. It may also be beneficial for the cover layer 140A to be temperature rated and able to resist temperatures 380-400 degrees Celsius or greater. The cover layer 140A may also be made of a polyimide film with a high-temperature rating.

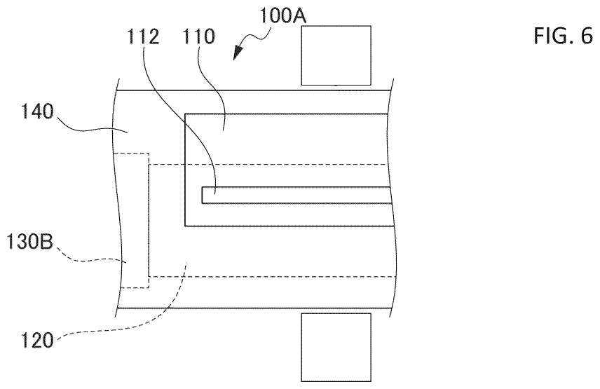

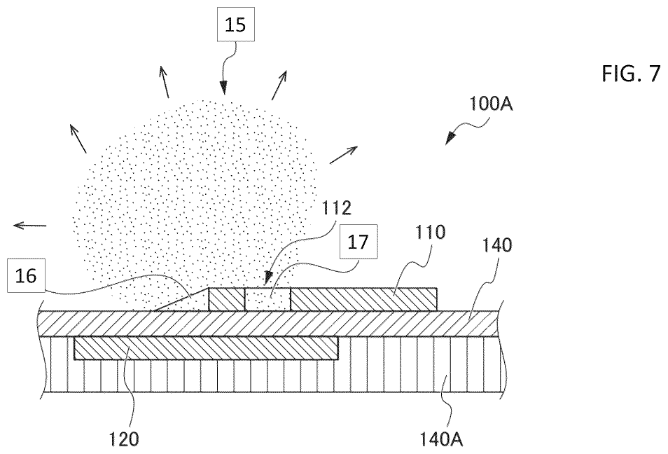

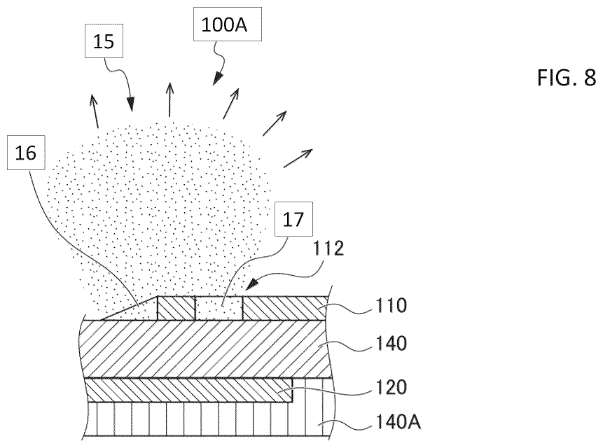

Next, a plasma discharge portion 100A according to a modification of the present embodiment will be described. FIG. 6 is a partial plan view of the discharge portion 100A according to the modification, FIG. 7 is a schematic cross-sectional view thereof, and FIG. 8 is a partial enlarged view thereof. In the modified example, the first electrode 110 and the second electrode 120 are formed on both surfaces of the dielectric layer 140 in the same manner as previously described. The positional relationship between the first electrode 110 and the second electrode 120 across the substrate 140 is also the same as previously described.

In the modified example, the structure of the discharge portion 100A is different in that the groove 112 is provided along the section of the first electrode 110 that overlaps the second electrode 120. In particular, as clearly shown in FIG. 8, the groove 112 is provided with the first electrode 110 removed so as to reach the substrate 140, which is a dielectric, at a distance from the side edge of the first electrode 110. In some embodiments, the depth of the groove 112 may not fully reach the dielectric 140. Therefore, when a high-frequency voltage, as previously discussed, is applied between the first electrode 110 and the second electrode 120, the groove 112 acts to form a plasma region 17 in addition to the plasma region 16, offering the same properties as the plasma region 11 described above.

In the modified example, since more active species 15 are generated by the atmospheric pressure low-temperature plasma generated in the extended plasma regions 16 and 17, the cleaning performance for the ambient air can be improved. The distance between the side edge section of the first electrode 110 closest to the plasma region 16 and the groove section 112 may be determined based on a multitude of interacting variables, including but not limited to the size of the dielectric layer 140, the material of the dielectric layer 140, the specification of the high-frequency voltage applied between the electrodes, and the dimensions of the electrodes 110, 120.

In some embodiments, the bottom of the cover layer 140A can be adhered to a structure intended for use in air conditioning or air filtering applications, such as in a window air conditioning unit or in HVAC ducts. Embodiments are intended to include any mechanism of bonding, including by mechanical means, like rivets, bolts, or chemical means, like adhesive, tape, epoxy, or glue, or any other known related or later developed apparatus. It might also be beneficial for the bonding material to be temperature rated and to be able to keep a high bonding strength at temperatures above room temperature for long periods of time. It might also be beneficial for the bonding material to be chosen to not interfere with the discharge from the plasma regions 11,12,16, and 17. In some embodiments, the cover layer 140A can be used as a bonding material to bond the plasma actuator discharge portion 100A to a structure intended for use in air conditioning or air filtering applications, such as in a window air conditioning unit or in HVAC ducts.

### D. Variations of Embodiments

Embodiments are intended to include or otherwise cover any methods and apparatus for creating stable cold plasma. Some of these embodiments are directed to generating stable cold plasma under atmospheric conditions where ambient fluid and small biological particles enter and interact with the plasma. In some of these embodiments, some or all of the small biological particles are sterilized by interaction with the stable cold plasma. However, other embodiments generate stable cold plasma for interaction with other fluids, such as fluids that include various gas species and concentrations.

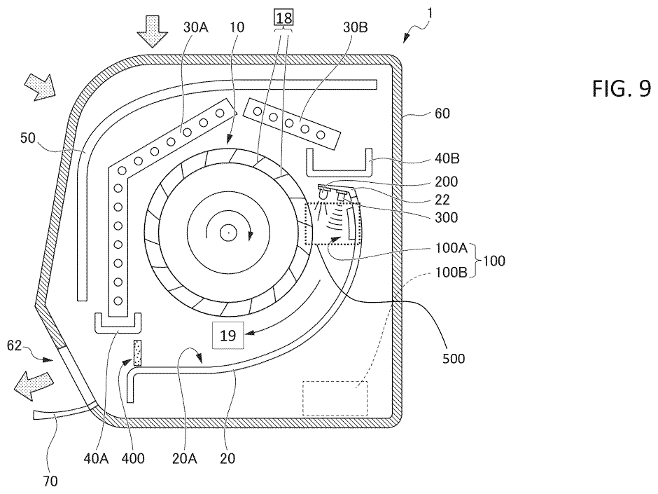

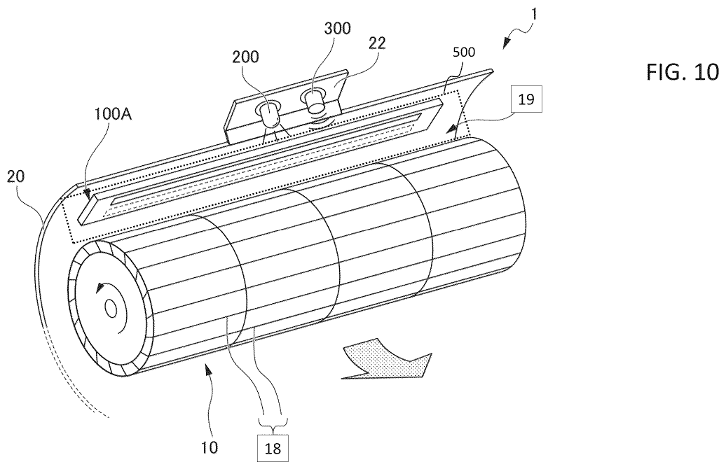

In some embodiments, an air conditioner 1 can be provided with the plasma actuator apparatus 100. FIG. 9 describes the air conditioner 1 and the plasma actuator 100 in a cross-sectional schematic view of an exemplary embodiment, and FIG. 10 describes the air conditioner 1 and the plasma actuator 100 in a perspective schematic view.

In the air conditioner 1, an air blower fan 10, an air guide plate 20, heat exchangers 30A and 30B, drain pans 40A and 40B, and an intake filter 50 are generally accommodated in a cabinet 60. The air blower fan 10 is configured as a cross-flow fan having a plurality of fan blades 18, and has a function of taking outside air into the cabinet 60 and generating an air flow for discharging the air after heat exchange to the outside. The air guide plate 20 has a function of a guide for defining an air passage 19 for efficiently discharging the air flow by the air blower fan 10 from the air blower port 62 of the cabinet 60 to the outside with the air blower fan 10, and is formed of a metal plate or a resin molded product.

In some embodiments, a lack of insulation, whether it be material or distance, between the fan blades 18 and the plasma discharge portion 100A can lead to electrical arcing, which can degrade materials over time. In order to mitigate this issue, it may be beneficial to increase the insulation between the components by added insulating layers or distance between them. In some embodiments, the fan blades 18 are coated by an insulating layer to prevent arcing. In some embodiments, the fan blades 18 are made of plastic or a different insulating material.

The heat exchangers 30A and 30B are connected to a refrigerant circuit of an outdoor unit (not shown) and have a bent piping structure provided with a large number of fins. The heat exchangers 30A and 30B cool the air taken in from the outside by the heat of vaporization of the refrigerant compressed by the compressor of the outdoor unit. The drain pans 40A, 40B serve as receptacles for condensed water adhering to the heat exchangers 30A, 30B. The intake filter 50 has a function of removing foreign matter such as dust from air taken into the cabinet 60 of the indoor unit from the outside. The air blowing port 62 of the cabinet 60 is provided with a wind direction plate 70 for adjusting the direction of air discharged to the outside.

The air conditioner 1 of FIG. 9 described above is provided with a plasma actuator apparatus 100, comprising of 100A and 100B, an ultraviolet light lamp 200, an ultrasonic oscillator 300, and an ozone filter 400. These elements decompose and remove harmful components and odor components from the air to be cooled or heated in the air conditioner 1. FIG. 10 is a perspective view schematically showing the configuration of the air conditioner 1 around these elements. The discharge space 500 is the volumetric region in which the plasma discharge portion 100A is placed or any other modification of 100A or structure meant to hold one or multiple discharge portions 100A.

The plasma actuator apparatus 100 has an electrode formed of a pair of belt-like conductive materials to which a voltage signal varying with time is supplied from a power supply unit 100B and has a function of generating atmospheric pressure low-temperature plasma by discharge generated between the electrodes in the discharge portion 100A. As shown in FIGS. 9 and 10, the plasma actuator apparatus 100 of the present embodiment is formed as a flat and elongated belt-like member having the belt-like conductive material electrodes, and is provided so as to face the air passage 19 (a space between the air blowing fan 10 and the inner surface of the air guide plate 20) of the airflow in the cabinet 60 generated by the air blowing fan 10 so that its length is along the axial direction of the air blowing fan 10, for example, as shown in FIG. 10. With such a configuration, mixing of various active species generated by the atmospheric pressure low-temperature plasma of the plasma actuator apparatus 100 with the air flowing in the air passage 19 is promoted as described previously while effectively utilizing the space in the cabinet 60, and the cleaning action of the air is promoted.

The ultraviolet lamp 200 and the ultrasonic oscillator 300 are mounted or adhered so as to face the air passage 19 through a support frame 22 attached to the upper edge of the air guide plate 20. However, the mounting location and the mounting technique of the ultraviolet light lamp 200 and the ultrasonic oscillator 300 are not limited to the above example as long as they effectively act on the air flowing in the air passage 19.

With respect to the above, the surface of the fan blade 18 provided in the air blower fan 10 and the surface of the air guide plate 20 facing the air blower fan 10 may be subjected to a surface treatment to increase the reflectance of light, such as a mirror finish. In this way, energy generated from the atmospheric pressure low temperature plasma on the surface of the fan blade 18 and the surface of the air guide plate 20 is scattered in the air passage 19, so that the generation of various active species is promoted.

The ozone filter 400 shown in FIG. 9 is provided near the exhaust port 62 of the cabinet 60 in order to remove ozone (03) contained in the active species generated by the atmospheric pressure low temperature plasma. Thus, the concentration of ozone discharged from the air conditioner 1 is prevented from exceeding the regulation value by the related laws and regulations, and the odor peculiar to ozone is suppressed. The ozone filter 400 can be suitably constituted by using a material equivalent to an exhaust filter used in a copying machine.

In some embodiments, the heat exchangers 30A and 30B cause the humidity of the air inside the air conditioner unit 1 to increase as well as the walls to develop condensation. The humidity and condensation may require the parameters of the plasma actuator 100 to be modified in order to mitigate potential problems, like shorting of electrical components or undesirable electrical arcing.

The ultraviolet lamp 200 and the ultrasonic oscillator 300 described above are not explicitly shown in the following figures, but they can be added to any of the following embodiments. In addition, they can be placed in almost any position and facing in almost any desired direction, including towards or away from the flow of incoming fluid or ambient air.

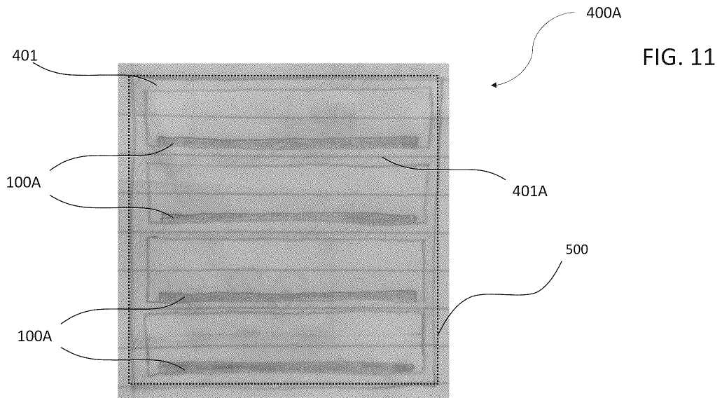

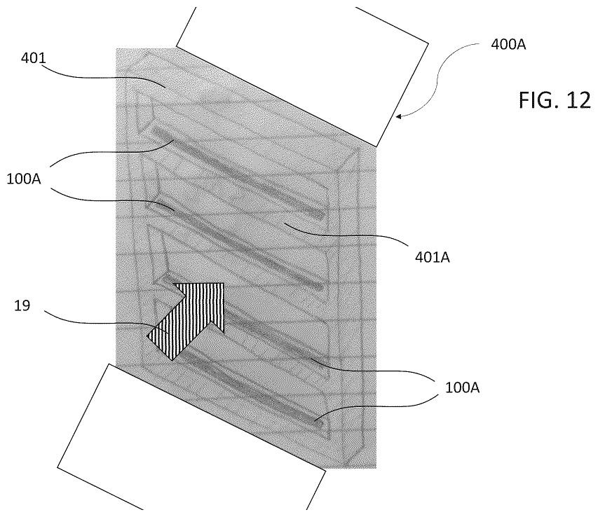

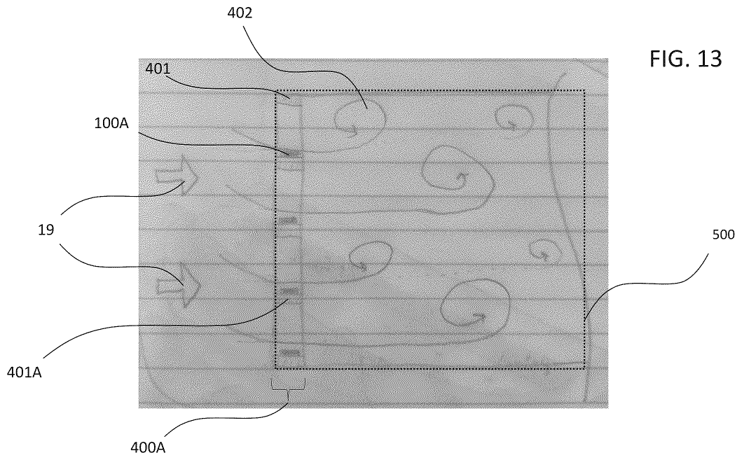

FIGS. 11 through 15 describe another exemplary embodiment of the discharge space 500 of the air conditioning unit described in FIGS. 9 and 10. The discharge portion 100A is replaced by a different structure 400A that includes multiple plasma actuators apparatus 100 instead of just one. In FIG. 11, the legs 401A of the support structure 401 acts to hold the individual plasma actuator apparatus 100 in their desired positions. In FIGS. 12 and 13, the position of the support structure 401 is shown relative to the stream of air 19 through the air conditioning unit at the location of the discharge space 500. The support structure 401 is shown to have a rectangular shape, however the structure 401 may have any shape intended to fit the discharge space 500 or any other space intended for the structure 401.

One of the effects of a plasma actuator apparatus 100 described above is the formation of a vortex in a stream of air. FIG. 13 shows one possible structure of a vortex formed by a plasma actuator 100A, in this case a travelling transverse vortex 402. In some embodiments, the plasma actuator apparatus 100A are pulsed to allow for a repeated formation of traveling transverse vortices 402. In FIG. 13, the plasma actuator discharge portions 100A, connected to power supply units 100B, are pulsed at two separate times in two groups, where each group consists of alternating plasma actuators apparatus 100. However, embodiments are intended to cover any pattern of pulsing the plasma actuators apparatus 100 to produce travelling transverse vortices 402.

In FIGS. 11 through 13, there are four plasma actuator discharge portions 100A included in the discharge structure 400A, however embodiments are intended to include any number of plasma actuator discharge portions 100A in order to properly fit an intended space. Embodiments are also intended to include variability in the selected parameters of each plasma actuator 100A in the discharge structure 400A.

In some embodiments with multiple plasma actuator discharge portions 100A, each plasma actuator discharge portion 100A is connected to a power supply unit 100B. In some other embodiments with multiple plasma actuator discharge portions 100A, the plasma actuator discharge portions 100A are connected to a single power supply unit 100B. In some embodiments, the power supply unit 100B may need to be modified to accommodate the increase in power required by multiple plasma actuator discharge portions 100A as opposed to just one plasma actuator discharge portion 100A.

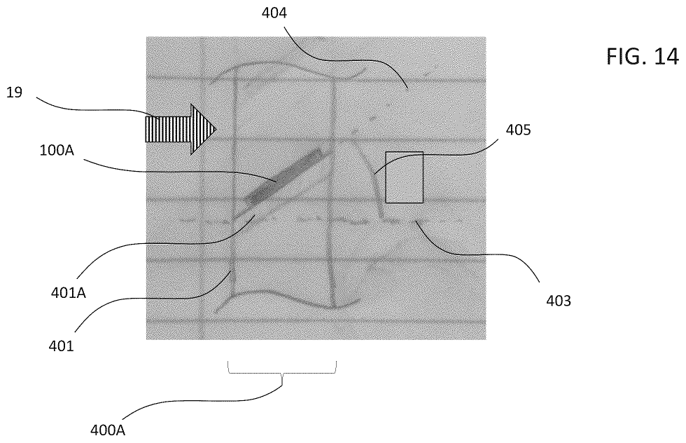

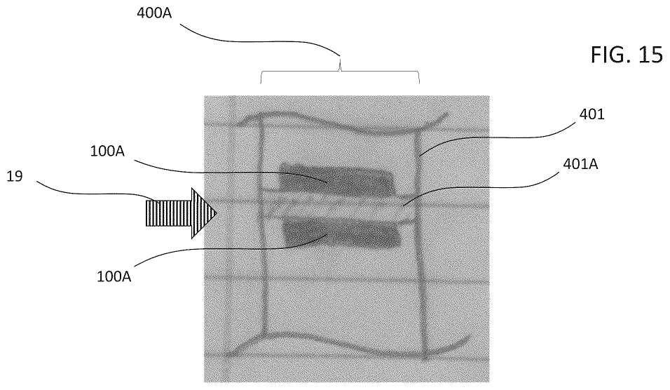

FIGS. 14 and 15 describe some basic modifications to the above outlined discharge structure embodiment 400A. In FIG. 14, a change to the angular position of the plasma actuator 100A can be made by modifying the legs 401A of the support structure 401. It may be beneficial to define a radial angle 405, which is the angle between the horizontal axis 403 that is parallel to the stream of air 19 through the discharge structure 400A and the axis 404 that exists in the plane of the plasma actuator 100A and is perpendicular to the length direction of the plasma actuator 100A. The modification to the radial angle 405 may allow different vortex structures to form, changing the rate of mixing of the radicals formed by the plasma actuator 100A with the air.

FIG. 15 describes the placement each plasma actuator 100A on the support structure 401. In some embodiments, the bottom of each plasma actuator can be adhered to either the top or bottom of each leg 401A of the support structure 401. In some embodiments, each leg 401A of the support structure 401 can hold two plasma actuators 100A, one on the top and the other on the bottom.

In some embodiments, the legs 401A of the support structure 401 may not be included. In some other embodiments, the legs 401A of the support structure 401 may have a different shape to that described in FIGS. 11 through 15. In some embodiments, the plasma actuator discharge portion 100A can be adhered to the support structure 401 on its sides near the electrode terminals 130B and 130A.

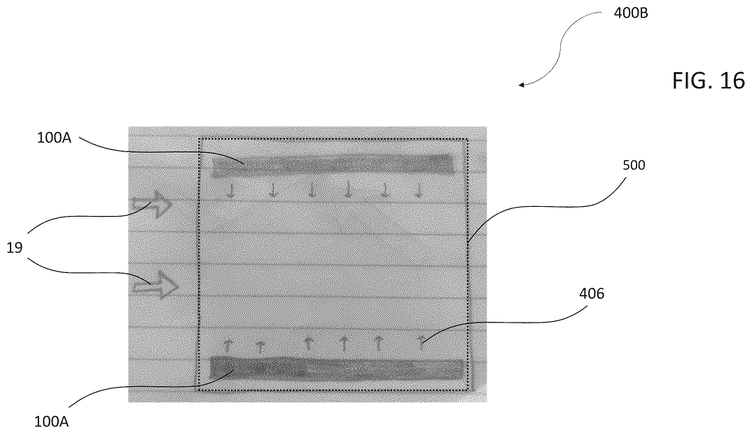

FIGS. 16 through 22 describe a third exemplary embodiment of the discharge space 500 of the air conditioning unit described in FIGS. 9 and 10. The discharge portion 100A is replaced by a different structure 400B that includes multiple plasma actuator discharge portions 100A instead of just one and is different to the structure 400A described in FIGS. 11 through 15. FIG. 16 shows the position of the plasma actuator discharge portions 100A in a top down view relative to the stream of air 19. The position of the electrodes 110,120 on the dielectric layer 140 that comprise each of the plasma actuator discharge portions 100A decides the direction of the induced flow. FIGS. 16 through 18 describe the case of two plasma discharge portions 100A positioned near the sides of the discharge space 500 with the electrodes 110,120 positioned on the dielectric layer 140 of each plasma actuator discharge portions 100A so that the induced flows 406 point inwards towards the center of the discharge space 500.

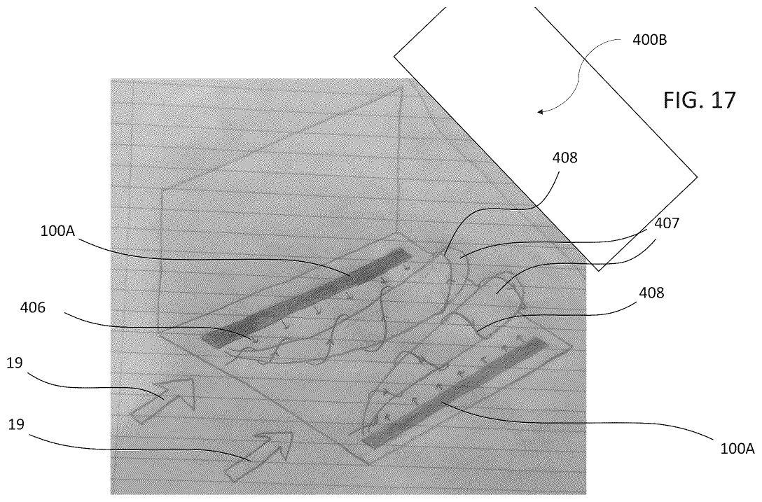

FIG. 17 describes the effect that the configuration described in FIG. 16 will have on the movement of air 19 through the discharge structure 400B. The induced flow 406 from each plasma actuator discharge portions 100A forms streamwise vortices 407, which are vortices that travel around an axis parallel to the stream of air 19. The trajectory 408 describes the flow of the outer surface of each streamwise vortex 407. Streamwise vortices have been found to greatly and efficiently mix the composition of fluid as they create strong shear fields within each vortex, which are the driving forces behind mixing.

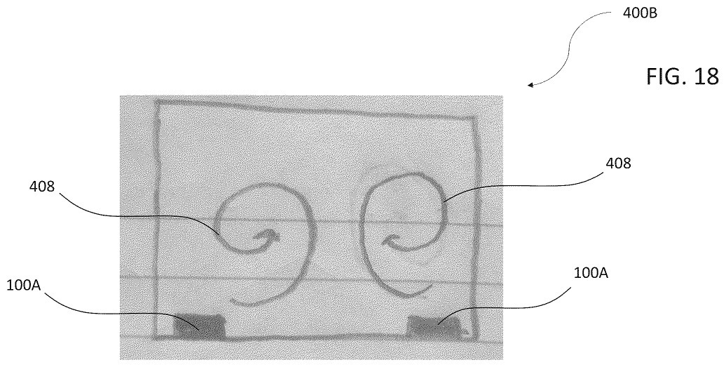

Since the induced flow 406 of each plasma actuator discharge portions 100A points in opposite directions towards the center of the discharge space 500 shown in FIG. 16, the flow at the center will be diverted. FIG. 18 shows this process along the length of the discharge space 500 shown in FIG. 16. The general flow can be described by a lobed flow, where the air travels up in the center of the discharge space 500 shown in FIG. 16 and down on the sides of the discharge space 500 shown in FIG. 16 with a clockwise-pointing vortex to the right of the center of the discharge space 500 shown in FIG. 16 and a counterclockwise-pointing vortex to the left of the center of the discharge space 500 shown in FIG. 16.

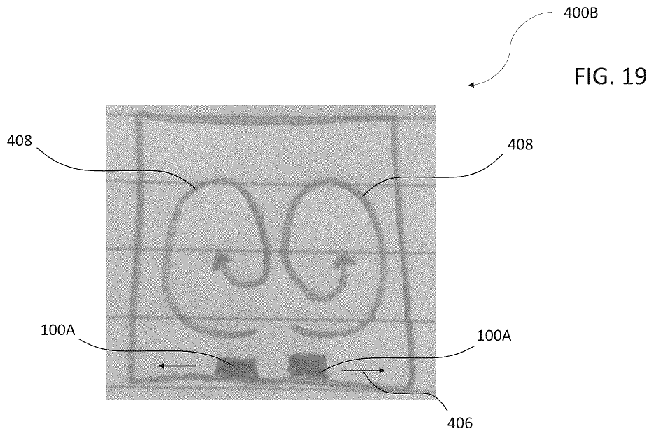

FIGS. 19 through 22 describe some basic modifications to the above outlined discharge structure embodiment 400B. In FIG. 19, the plasma actuator discharge portions 100A are flipped so that their induced flows 406 point away from each other and the plasma actuator discharge portions 100A are moved toward the center of the discharge space 500 shown in FIG. 16. This will create a different lobed flow configuration where the air travels down in the center of the discharge space 500 shown in FIG. 16 and up on the sides of the discharge space 500 shown in FIG. 16 with a counterclockwise-pointing vortex to the right of the center of the discharge space 500 shown in FIG. 16 and a clockwise-pointing vortex to the left of the center of the discharge space 500 shown in FIG. 16.

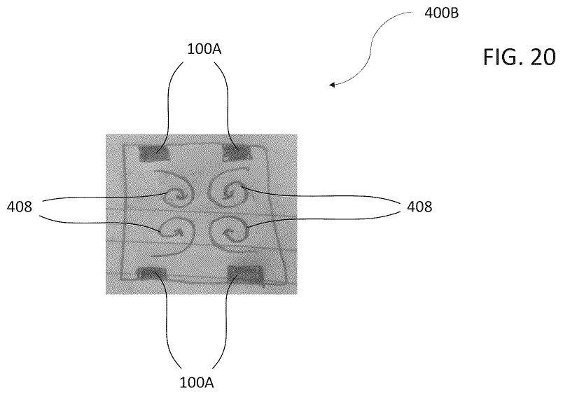

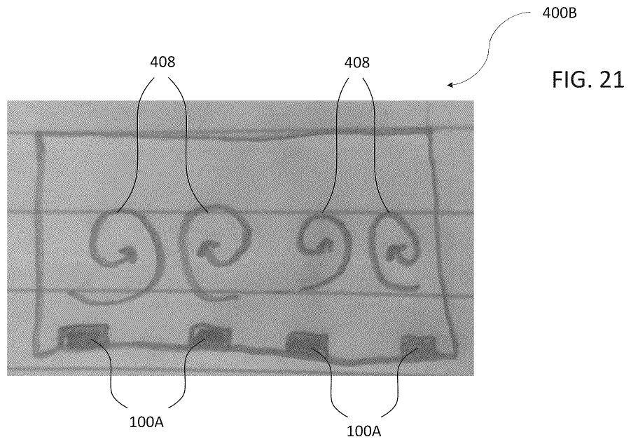

FIG. 20 describes a possible addition of downward-facing plasma actuator discharge portions 100A in the embodiment 400B. In some embodiments, the downward facing plasma actuator discharge portions 100A can form new streamwise vortices 408. In FIG. 21, there are four plasma actuator discharge portions 100A on the bottom of the discharge space 500 shown in FIG. 16 that can form four streamwise vortices 408, however, embodiments are intended to include any number of plasma actuator discharge portions 100A positioned anywhere in the discharge space 500 shown in FIG. 16 of the structure 400B to form streamwise vortices 408.

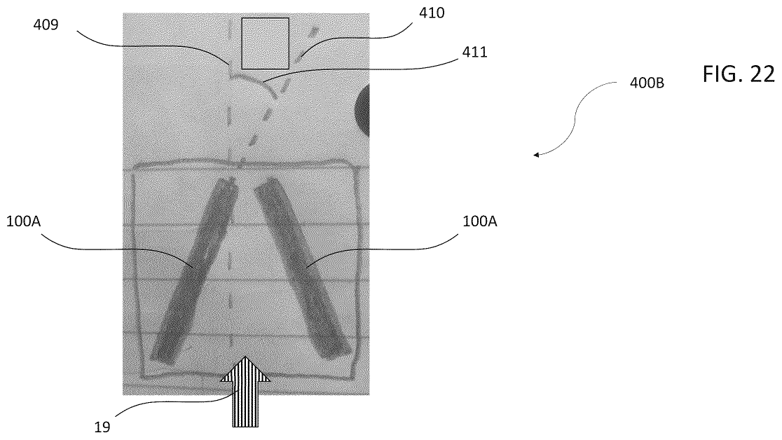

FIG. 22 describes a change to the angular position of the plasma actuators 100A in the structure 400B. It may be beneficial to define an azimuthal angle 411, which is the angle between the axis 409 parallel to the stream of air 19 and the axis 410 that defines the lengthwise direction of the plasma actuator discharge portion 100A. Changing this angle may allow for the same streamwise matrix formation, but a more effective mixing of radicals created by the plasma actuators 100A and the air 19.

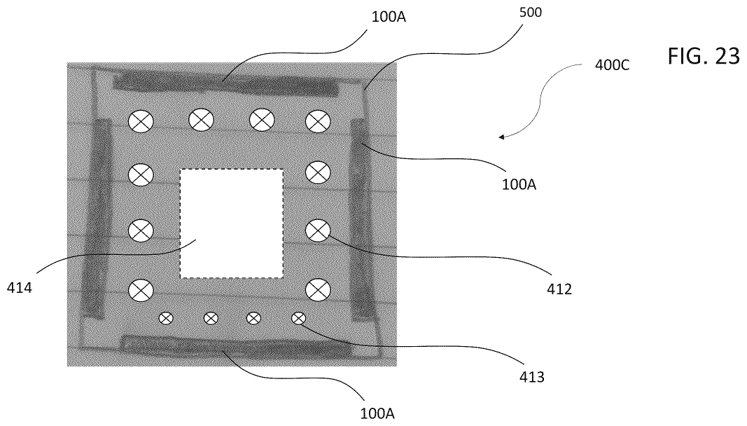

In some embodiments, the plasma actuator apparatus 100 can be provided as an addition to an existent HVAC system. FIGS. 23 through 25 describe a fourth exemplary embodiment of the discharge space 500 of the air conditioning unit described in FIGS. 9 and 10. The discharge portion 100A is replaced by a different structure 4000 that includes multiple plasma actuator discharge portions 100A instead of just one and is different to the structures 400A and 400B described in FIGS. 11 through 22.

FIG. 23 is a front schematic view of an exemplary embodiment 4000 with plasma actuator discharge portions 100A on all four sides of the discharge space 500. Three of the plasma actuator discharge portions 100A induce a strong flow 412 in the direction of the air flow, while one of the plasma actuator discharge portions 100A induces a weak flow 413 in the direction of the air flow. This can be accomplished by changing the parameters of one of the plasma actuator discharge portions 100A, such as the AC frequency. The difference in induced flows creates a shear field in the region 414, here called the vortex region.

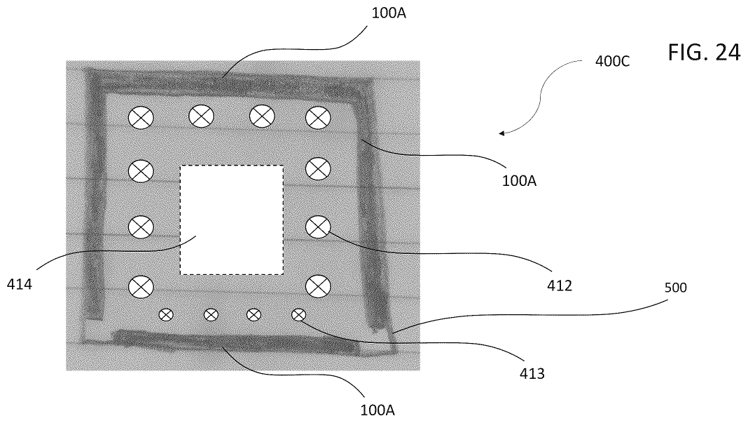

FIG. 24 describes a modification to the structure 4000 described in FIG. 23. In some embodiments, one of the plasma actuator discharge portions 100A can be shaped in the form of a U, replacing three of the plasma actuator discharge portions 100A in FIG. 23. The U shaped plasma actuator discharge portion 100A creates a strong induced flow 412 in the direction of the air flow, and the other plasma actuator discharge portion 100A induces a weak flow 413 in the direction of the air flow, creating a shear field and a vortex region 414.

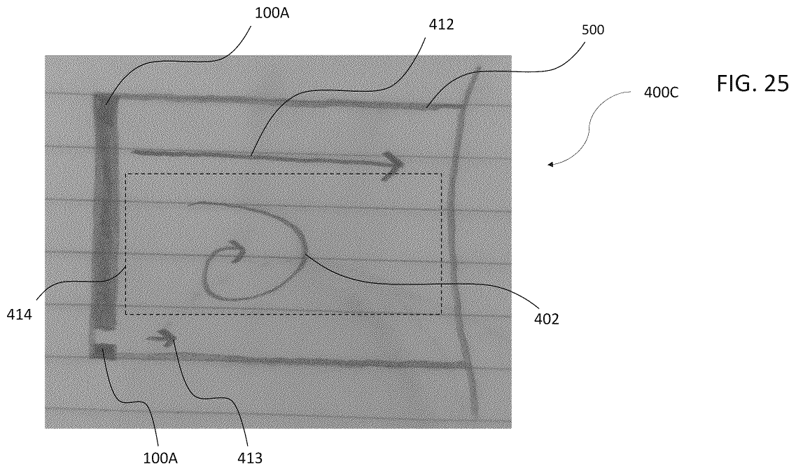

FIG. 25 is a side schematic view of the plasma actuator structure 4000 and describes the vortex region 414 in more detail. The plasma actuators 100A create the strong induced flow 412 above and the weak induced flow 413 below, which can cause transverse vortices 402 to form in the vortex region 414.

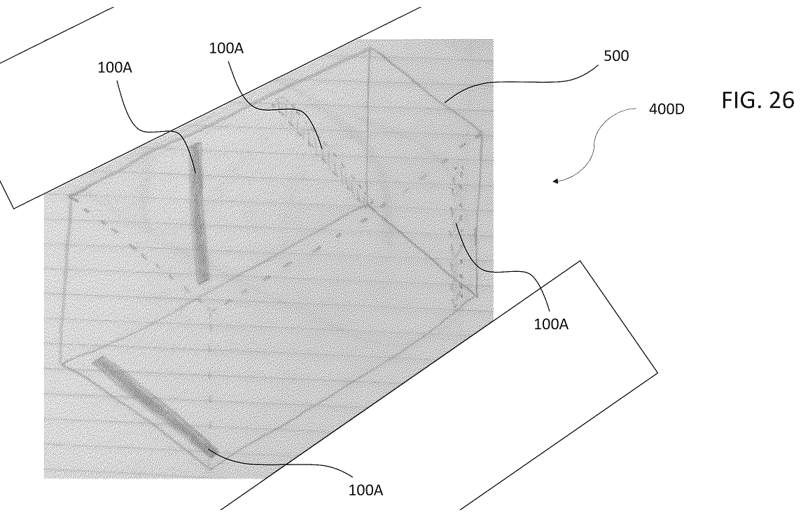

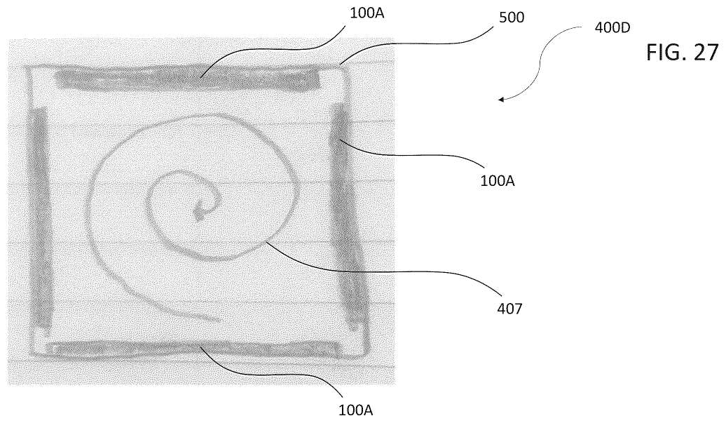

FIGS. 26 and 27 describe a fifth exemplary embodiment of the discharge space 500 of the air conditioning unit described in FIGS. 9 and 10. The discharge portion 100A is replaced by a different structure 400D that includes multiple plasma actuators apparatus 100 instead of just one and is different to the structures 400A, 400B, and 4000 described in FIGS. 11 through 25.

FIG. 26 shows a perspective view of an exemplary embodiment of the discharge structure 400D. The plasma actuator discharge portions 100A are placed on all four sides of the discharge space 500. There is some distance in the length direction of the discharge space 500 between each plasma actuator discharge portion 100A so that the plasma actuator discharge portions 100A alternate clockwise around the discharge space 500. In some embodiments, the plasma actuator discharge portions 100A alternate counterclockwise around the discharge space 500.

FIG. 27 is a front schematic view of the plasma actuator structure 400D and describes the effect of the alternation of the plasma actuator discharge portions 100A around the discharge space 500. The alternating placement of the plasma actuator discharge portions 100A creates a streamwise vortex 407 along the discharge space 500.

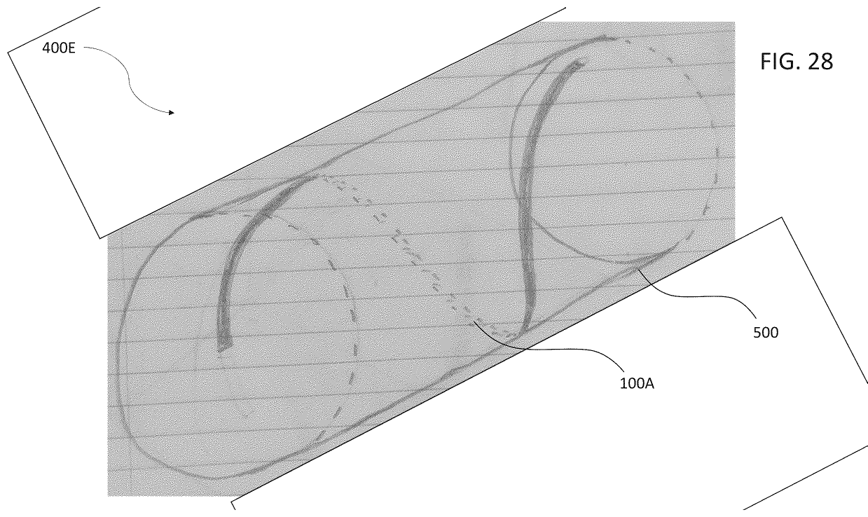

FIG. 28 describes a sixth exemplary embodiment of the discharge space 500 of the air conditioning unit described in FIGS. 9 and 10. The discharge portion 100A is replaced by a different structure 400E that includes a single plasma actuator apparatus 100 and is different to the structures 400A, 400B, 4000 and 400D described in FIGS. 11 through 27. In some embodiments, the discharge space 500 may be cylindrical. In this case, the plasma actuator discharge portion 100A can be formed into a spiral shape in either the clockwise or counterclockwise direction going down the discharge space 500. In some embodiments, the discharge space 500 may be a different shape than a cylinder, but the plasma actuator discharge portion 100A can still be formed into a spiral shape.

Embodiments 400A, 400B, 4000, 400D, and 400E and modifications to the mentioned embodiments can be provided in an air conditioning unit similar to or the same as the air conditioner unit 1 described in FIGS. 9 and 10. Some of or all of the components of embodiments 400A, 400B, 4000, 400D, and 400E can fit into the discharge space 500 outlined in FIGS. 9 and 10. Embodiments 400A, 400B, 4000, 400D, and 400E and modifications to the mentioned embodiments can also be provided in an HVAC system. To prevent the escape of ozone generated by the plasma actuator apparatus 100 in an HVAC system, the components of embodiments 400A, 400B, 4000, 400D, and 400E can be provided with an ozone filter with similar properties or the same properties as the ozone filter 400 described above.

What is claimed is:

1. A plasma generator for generating atmospheric pressure, low temperature plasma, comprising:
a dielectric layer, having a dielectric constant, that is elongated in a longitudinal direction, and that extends 0.01 mm-2 mm in a thickness direction that is perpendicular to the longitudinal direction, the dielectric layer defining a first planar surface and an opposing second planar surface that are separated in the thickness direction;
a first electrode that is disposed along a first portion of the first planar surface;
a second electrode that is disposed along a second portion of the second planar surface, such that at least a part of the first and second portions are separated in the longitudinal direction of the dielectric layer; and
a power supply configured to supply electrical power to the first and second electrodes at a predetermined voltage and frequency, such that, based on the dielectric constant of the dielectric layer, atmospheric pressure, low temperature plasma is generated adjacent each of the first and second electrodes and along the first and second surfaces of the dielectric layer other than the first and second portions.

2. The plasma generator of claim 1, wherein the power supply is configured to generate the atmospheric pressure, low temperature plasma so as to change a flow of fluid into a direction generally in the longitudinal direction of the dielectric layer.

3. The plasma generator of claim 1, wherein the dielectric layer extends 0.02 mm-0.08 mm in the thickness direction.

4. The plasma generator of claim 3, wherein the dielectric layer extends approximately 0.05 mm in the thickness direction.

5. A plasma generator for generating atmospheric pressure, low temperature plasma, comprising:
a dielectric layer, having a dielectric constant, that is elongated in a longitudinal direction, and that extends 0.01 mm-2 mm in a thickness direction that is perpendicular to the longitudinal direction, the dielectric layer defining a first planar surface and an opposing second planar surface that are separated in the thickness direction;
a first electrode that is disposed along a first portion of the first planar surface;
a second electrode that is disposed along a second portion of the second planar surface, such that at least a part of the first and second portions are separated in the longitudinal direction of the dielectric layer;
an insulator that covers the second electrode and the second planar surface other than the second portion; and
a power supply configured to supply electrical power to the first and second electrodes at a predetermined voltage and frequency, such that, based on the dielectric constant of the dielectric layer, atmospheric pressure, low temperature plasma is generated adjacent the first electrode proximate along the first surface of the dielectric layer other than the first portion.

6. The plasma generator of claim 5, wherein the power supply is configured to generate the atmospheric pressure, low temperature plasma so as to change a flow of the fluid into a direction generally in the longitudinal direction of the dielectric layer.

7. The plasma generator of claim 5, wherein the dielectric layer extends 0.02 mm-0.08 mm in the thickness direction.

8. The plasma generator of claim 7, wherein the dielectric layer extends approximately 0.05 mm in the thickness direction.

9. The plasma generator of claim 5, wherein the first electrode defines a groove in the longitudinal direction of the dielectric layer, so as to define first and second separated sections, and such that atmospheric pressure, low temperature plasma is generated proximate the groove.

10. An apparatus for receiving a fluid, communicating atmospheric pressure, low temperature plasma to the fluid to result in treated fluid, and supplying the treated fluid to a space, the apparatus comprising:
an plasma generator for generating atmospheric pressure, low temperature plasma, the plasma generator including:
a dielectric layer, having a dielectric constant, that is elongated in a longitudinal direction, and that extends 0.01 mm-2 mm in a thickness direction that is perpendicular to the longitudinal direction, the dielectric layer defining a first planar surface and an opposing second planar surface that are separated in the thickness direction;
a first electrode that is disposed along a first portion of the first planar surface;
a second electrode that is disposed along a second portion of the second planar surface, such that at least a part of the first and second portions are separated in the longitudinal direction of the dielectric layer;
an insulator that covers the second electrode and the second planar surface other than the second portion; and
a power supply configured to supply electrical power to the first and second electrodes at a predetermined voltage and frequency, such that, based on the dielectric constant of the dielectric layer, atmospheric pressure, low temperature plasma is generated adjacent the first electrode proximate along the first surface of the dielectric layer other than the first portion;
a housing that houses the plasma generator and that defines an entry aperture for enabling entry of the received fluid, and an exit aperture for enabling exit of the treated fluid into the space; and
a fan configured to direct the received fluid to the plasma generator and through the exit aperture into the space.

11. An apparatus of claim 10, further comprising a heat exchanger that changes a temperature of the received fluid.

12. An apparatus of claim 10, further comprising a UV emitter and an ultrasonic oscillator that are disposed proximate the plasma generator and configured to facilitate generation of the atmospheric pressure, low temperature plasma.

13. An apparatus of claim 10, further comprising an ozone filter disposed proximate the exit aperture to impede passage of ozone in the treated fluid.
